(19) United States
(12) Patent Application Publication
(10) Pub. No.: US 2001/0046704 A1
(43) Pub. Date: Nov. 29, 2001

(54) Methods and compositions to activate genes in cells

(76) Inventor: Gary Kent McMaster, Ann Arbor, MI (US)

Correspondence Address: Mehdi Ganjeizadeh, Warner-Lambert Company, 2800 Plymouth Road, Ann Arbor, MI, 48105, US

(21) Appl. No.: 09/864,001

(22) Filed: May 23, 2001

Related U.S. Application Data
(60) Provisional application No. 60/206,768, filed on May 23, 2000.

(51) Int. Cl. C12N 15/01; C12N 5/06

(52) U.S. Cl. 435/441; 435/325

(57) ABSTRACT

The present invention relates to methods and compositions to activate or modify the expression or activity of selected genes or gene pathways in cells. The invention more particularly relates to methods of preparing cells that express selected genes or gene pathways by mutagenesis in vitro, ex vivo, or in vivo. The invention more preferably discloses methods of modifying a regulator gene or sequence in a cell, so that the activity or expression of a selected gene or gene product is modified, without the structure of said gene product being altered. This invention can be used to produce improved cells for use in various areas such as screening, production, genetic analysis, cloning, cell therapy products, etc.

### FIELD OF THE INVENTION

The present invention relates to methods and compositions to activate or modify the expression or activity of selected genes in cells. The invention more particularly relates to methods of preparing cells that express selected genes by mutagenesis in vitro, ex vivo, or in vivo. The invention more preferably discloses methods of modifying a regulator gene or sequence in a cell, so that the activity or expression of a selected gene or gene product is modified, without the structure of said gene product being altered. This invention can be used to produce improved cells for use in various areas such as screening, production, genetic analysis, cloning, cell therapy products, etc.

### BACKGROUND OF THE INVENTION

The regulation or activation of genes or gene pathways has many implications in research, development, and the identification of new therapeutic areas. In particular, the ability to provide cells that express a selected phenotype is very useful in research and development, for the screening of the efficacy, selectivity, or activity of compounds. Generally, the provision of such cells require knowledge and availability of selected genes that are cloned and introduced into cells to ensure expression thereof or the production of a desired phenotype.

### SUMMARY OF THE INVENTION

The present invention now provides alternative methods to regulate or activate genes or gene pathways and to prepare cells having selected phenotype. The invention is based on mutagenesis and selection steps, and avoids the need for and the use of cloned genes.

A particular aspect of the present invention resides in a method of causing expression or activation of a selected gene or gene pathway in a cell, comprising exposing a cell population to at least a mutagenic agent or condition, and selecting the cells in the exposed population that express the selected gene or have an activated selected gene.

The method can be used to cause expression or activation of selected genes encoding receptor molecules (or sub-units thereof), such as a nuclear or a membrane receptor, in particular a receptor for a neurotransmitter, a hormone, a growth factor, a cytokine or a trophic factor; channels (or sub-units thereof); cytoplasmic polypeptides, etc.

In a more particular embodiment of this invention, the expression or activation of the selected gene is a result of one or several mutations in a gene or sequence in the cell that regulates the expression or activity of the selected gene in said cell.

In a particular variant, the method of the present invention is a method of causing overexpression of the selected gene, ie, expression at a level above the basal level in the cell, preferably at least 2 times above said basal level, even more preferably at least 5 to 10 times above said basal level.

In another particular embodiment, the method of the present invention is a method of causing expression of a selected gene, which is not expressed in the cells of a selected cell population. In this embodiment, the mutagenic treatment leads to modification(s) in regulatory sequences, which control the expression of the selected gene, such as promoters, silencers, enhancers, transcription factors, etc.

These embodiments are advantageous since they allow the preparation, from a selected cell population, of a cell (or cell line) which expresses a selected gene and produces the selected gene product, without the need to clone said gene and prepare recombinant cells.

In still another embodiment, the method of this invention can be used to cause activation of the selected gene or gene product, by modifying a product involved in the signaling pathway of the gene product. Such product may be an adaptor, transducing molecule, kinase, phosphatase, etc. Such a modification leads, in a particular embodiment of the present invention, to cells in which the gene product is activated in the absence of a natural ligand or activator thereof.

The methods referred to above may be implemented using many different cells or cell types, including essentially all cultivable cells. These may be primary cells or established cell lines, of mammalian, prokaryotic, plant, or lower eukaryotic origin. In a particular embodiment, the cell contains the selected gene in its genome, but the gene is not expressed or activated. Furthermore, while the cells may be isolated and cultured in vitro or ex vivo for mutagenesis, whole organisms or tissues may be used as well, in vitro, ex vivo, or in vivo. In such a case, the mutagenized cells may be isolated from the treated tissue or organism.

As will be further described in this application, in performing the method of the present invention various mutagenic agents or conditions can be used, including chemical mutagens as well as physical treatments such as irradiation (eg, X-ray radiation, gamma radiation, ultraviolet light, etc.), either alone or in combinations.

Selection of the expressed or activated gene or resulting phenotype may be performed according to several techniques and strategies such as, more preferably, those using an antibody to the selected gene product, a signal transduction pathway reporter system, a functional assay and/or with a marker-driven response. In this regard, in a preferred embodiment, the cell population comprises cells that contain a reporter gene construct to allow selection of activated gene in said cells following mutagenic treatment thereof. The reporter gene construct may preferably comprise a reporter gene (ie, a nucleic acid encoding a product which can be detected) under the control of a transcriptional promoter that is activated in the presence of a molecule involved in the signaling pathway of the selected gene product.

More specific embodiments of this invention include a method of causing expression or activation of at least two selected genes in a cell, comprising:

(i) exposing a first cell population to at least a mutagenic agent or condition and selecting the cells in the first population that express a first selected gene or have an activated first selected gene,

(ii) exposing a second cell population to at least a mutagenic agent or condition and selecting the cells in the second population that express a second selected gene or have an activated second selected gene, and

(iii) fusing the cells selected in (i) and (ii) to produce a cell having two expressed or activated selected genes.

In a particular embodiment, the above dual method is used to prepare cells that express two selected gene products such as two nuclear or membrane receptors.

A more specific aspect of this invention resides in a method of causing expression of a selected gene encoding a membrane receptor in a cell, comprising exposing a cell population to at least a mutagenic agent or condition and selecting the cells in the exposed population that bind a ligand of the membrane receptor, said cells expressing the membrane receptor.

An aspect of this invention also comprises a method of causing activation of a receptor expressed in a cell so that the receptor is active without a ligand of said receptor, comprising exposing a cell population to at least a mutagenic agent or condition in the absence of a ligand of the receptor, and selecting the cells in the exposed population having an activated receptor, more preferably the cells that express a molecule involved in the signaling pathway of the activated receptor.

Another aspect of the present invention resides in a method of causing expression or activation of a selected phenotype in a cell, comprising exposing a cell population to at least a mutagenic agent or condition and selecting at least a cell in the exposed population that express the selected phenotype.

A further object of this invention is a method of screening for compounds that modulate a gene or a gene product activity in a cell, comprising (i) contacting candidate compounds with a cell, wherein said cell or an ancestor thereof has been exposed to at least a mutagenic agent or condition which causes expression or activation of said gene, and (ii) selecting the compound or compounds that modulate said gene activity in the cell.

The screening may be performed under conventional conditions, in any suitable device, such as plates. The screened compounds may be any isolated molecule or mixtures, as well as any combinatorial library of products. Selection of the compounds can be made using conventional methods as described above, using affinity reagents, reporter systems, and the like.

This invention also relates to a cell produced by the above methods, as well as compositions comprising the same and any use thereof, including for screening, production, cell therapy, and the like.

### DETAILED DESCRIPTION OF THE INVENTION

The present invention relates, generally, to methods and compositions to activate gene expression or activity in a cell, in vitro, ex vivo, or in vivo. The invention relates more particularly to methods of activating gene expression or activity in a cell using mutagenic agent or condition, in order to produce a desired phenotype or genotype. More preferably, the method comprises activation of an effector gene or sequence (eg, promoter, enhancer, silencer, transcription factor, etc.) that regulates the activity or expression of a selected gene in a cell. The activated cells may be cultured, replicated, and stored and used in many technological areas such as screening, production, therapy, etc.

As mentioned above, the present invention involves the mutagenic treatment of cells to cause activation or expression of a selected gene or gene pathway and the selection of the cells having the desired genotype or phenotype. The various steps and materials for performing the invention will be disclosed in more details below. It should be understood that the invention is not limited to these more specific embodiments.

The Cells

The methods of the present invention may be implemented using many different cells or cell types, in vitro, ex vivo, or in vivo. The cells may be isolated (eg, cell suspensions or cultures) or included in tissue samples or whole organisms. For in vitro or ex vivo uses, essentially all cultivable or isolated cells or tissues may be used, including primary cells or established cell lines of mammalian, prokaryotic, plant, or lower eukaryotic origin. In this regard, where the selected gene is a mammalian gene, the cell should be of mammalian origin. In a particular embodiment, the cell contains the selected gene in its genome, but the gene is not expressed or activated.

Typical and preferred cells are ES stem cells, CHO, CHO-K1-Gal5, Hela, Jurkat Cos-7, Cos-2, THP-1, HL-60, HepG-2, SY5Y, F9, 3T3, L-cells, Hek 292, Hek 293, ECV 304, U937, MCF-7, CV-1, Ins-1, A549, MGH-UI, etc.

Furthermore, in a particular embodiment, the cells should contain a reporter gene construct to allow a selection of expressed or activated gene or gene pathway in said cells following mutagenic treatment thereof. Such a reporter construct is disclosed later in the application. The reporter construct may be introduced into the cells either before, during or after mutagenesis, as described in a subsequent section of this application.

The cells can be maintained in any conventional culture medium such as, for mammalian cells, RPMI, EAGLE, DMEM and the like, for instance. Culture can be performed generally around 37° C. (for human cells), in the presence of conventional additives (antibiotics, amino acids, serum, etc.). The cells can be cultured and/or stored in any appropriate device (tubes, flasks, bottles, etc.). Cell viability and/or absence of contamination can be verified prior to carrying out the methods of this invention.

While the cells may be isolated and cultured in vitro or ex vivo for mutagenesis, whole organisms or tissues may be used as well, in vitro, ex vivo, or in vivo. In such a case, the mutagenized cells may be isolated from the treated tissue or organism.

The Mutagenic Agent or Condition

Mutagenesis can be performed using a wide variety of chemical and/or physical treatment(s), known to affect a cell's genome, in vitro, ex vivo or in vivo.

In this regard, numerous chemical mutagens have been described and used in the past, as described for instance by Russell et al., Biology of Mammalian Germ Cell Mutagenesis, Cold Spring Harbor Laboratory Press, 1990:271-285; Rinchik E. M., Trends in Genetics, 1991;7(1):15-21; or Marker et al., Genetics, 1997;145(2):435-443. For mutagenesis, cells are preferably treated with increasing amounts of the chemical mutagen(s) either in whole animals or in cell suspensions, to assess the cytotoxicity of the chemical mutagen(s). Typical doses have been described in the literature as cited above. For example, cell suspensions (eg, mouse ES stem cells) are exposed to doses of N-ethyl-N-nitrosourea (ENU) as high as 0.4 mg/mL for 2 to 3 hours, which results in more than 98% of the cells dying. Viable cells harbor a higher mutation frequency and induce or repress gene expression as demonstrated by DNA chip experiments (11,500 mouse genes were tested). Other mutagens include for instance intercalating agents, alkylating agents, strand breaking agents, mismatch-inducing agents, etc.

In a more particular embodiment, the mutagenic agent is a mutagen causing a genomic modification selected from strand break, mismatch, and mutation.

The strand break may consist of single-strand (interruption in one of the two strands) and/or double-strand break (interruption in both strands). Ionizing radiation directly causes double-stranded breaks, whereas enzymatic incision indirectly causes double-stranded breaks.

Mismatch refers to a nonconvalent interaction between two nucleic acids residing on a different polynucleic acid sequence, not following base-pairing rules.

The mutation comprises deletion (absence of one or more nucleotides), insertion (addition of one or more nucleotides) and/or substitution (replacement of one or more nucleotides by a molecule which is different).

Specific and preferred chemical mutagens include ENU, an alkylating agent that mainly causes base substitutions (of which many are GC to AT transitions, with some AT to TA transversions) in DNA (less frequently small deletions), and therefore allows for recovery of complete and partial loss, as well as gain, of function alleles and is at least 10 times more efficient in generating mutations than other agents (Lawley P. D., Effects of some chemical mutagens and carcinogens on nucleic acids, Prog. Nucleic Acid Res. Mol. Bio., 1966;5:89-131and Amanuma K., Takeda H., Amanuma H., and Aoki Y, Transgenic zebrafish for detecting mutations caused by compounds in aquatic environments, Nature Biotechnology, 2000;18:62-65). Other more preferred chemical mutagens are benzo(a) pyrene, 2-amino-3,8-dimethyllimidazo(4,5,f)quinoxaline (Amanuma et al., supra., 2000), N-methyl-N′-nitro-N-nitrosoguanidine, methyl methane sulfonate, procarbazine, procarbazine hydrochloride, acrylamide monomer, chlorambucil, melphalan triethylene melamine, cyclophosphamide, diethyl sulfate, ethyl methane sulfonate, urethane, 6-mercaptopurine, mitomycin-C, methylnitrosourea, etc.

In a particular embodiment of the present invention, the mutagenic treatment thus comprises exposing the cell or tissue or organism to a chemical mutagen under conditions sufficient to alter the genome of the cells. The conditions include exposure time and mutagen concentration sufficient to alter the cells.

As indicated, mutagenesis can also be performed using physical treatments, in particular by irradiation, either alone or in combination with chemical mutagens, for instance. Irradiation may be performed according to techniques and under conditions known in the art.

In this regard, the skilled artisan can adapt the quality and quantity of the ionizing radiation to cause the desired genetic effect on biological materials. In particular, the operating potential across the X-ray tube, the current used, any filters used, the distance from the X-ray source, and the total amount of exposure may be adjusted by the skilled person, depending on the selected gene, the biological material, and the irradiation conditions (cell suspension, tissue, organism, etc.). Typically, with an X-ray machine, a potential of 250 kV and 15 mA current can be used to alter biological properties of cells and thus their phenotypes. The biological effects of gamma irradiation are essentially the same as those of X-radiation. Operating conditions for the gamma irradiators further depend on the particular machine being used. In this regard, suitable devices for use in the present invention include the X-ray machine and cobalt and cesium gamma irradiators. These machines generate high-energy photons that ionize atoms in matter receiving radiation. When the recipient of radiation is a living cell or organism, the radiation alters its biochemical make-up and therefore the functional properties of the cell(s) (Storer J. B., Acute responses to ionizing radiation. Biology of the Laboratory Mouse, McGraw-Hill:New York, 1966 and Chan E. L., Irradiation, in Selected Methods in Cellular Immunology, Freeman & Co.: San Francisco, 1980:242-244).

The results of irradiation also depend on the target organism. Indeed, species can vary in their radiosensitivity. For example BALB/cJ is the most sensitive of the common laboratory mouse strains. The LD50 (dose of total body irradiation necessary to kill 50% of the experimental animals within 30 days) of a typical mouse strain is 940 roentgen units (R) as measured by ionization instruments called dosimeters. The radiosensitivity of various subpopulations of lymphocytes differs, too. B cells and most T cells are relatively radiosensitive, whereas primed T helper cells are quite radioresistant (Kettman J. and Dutton R. W., Proc. Nat. Acad. Sci., 1971;68:699 and Chan E. L., Henry C., J. Immunol., 1976;117:1132).

In a particular embodiment of the present invention, the mutagenic treatment thus comprises exposing the cell or tissue or organism to irradiation under conditions sufficient to alter the genome of the cells. The conditions include exposure time and current strength sufficient to alter the cells.

In a preferred procedure to irradiate whole animals, whole animals are individually confined in containers when receiving X-ray to ensure exposure at a constant dose rate. Following irradiation animals are kept under clean conditions and supplied with acidic drinking water to minimize death due to infection by environmental pathogens before preparing tissues and cells of interest (Selected Methods in Cellular Immunology, edited by B. B. Mishell and S. M. Shiigi, 1980) and cell sorting according to Parks D. R., Byran V. M., Oi V. T., and Herzenberg L. A., Antigen-specific identification and cloning of hybridomas with a fluorescence-activated cell sorter, Proc. Natl. Acad. Sci., 1979;76 :1962-1965 to select for cells expressing the desired gene and its protein product.

In a preferred procedure to irradiate single-cell suspensions, cell suspensions are washed and resuspended at a density of between about 10⁵ and 10⁸ cells/mL in balanced salt solution, placed in the irradiation chamber before delivering the desired dose of radiation (eg, X-ray). After dosing, cells are washed multiple times in balanced salt solution to remove toxic free radicals and their products resulting from irradiation.

The Selection of the Cells with the Selected Phenotype

After the cell(s) of interest have been mutagenized, the phenotype (or activated gene or gene pathway) can be selected according to several techniques. In particular, the activated gene product or selected phenotype may be identified by using:

(i) One or several affinity reagents specific for the activated or expressed gene product (such as antibodies or fragments or derivatives thereof, or any other ligand). Selection with affinity reagents is particularly suitable where the selected gene product is exposed at the surface of the cell, such as for instance membrane receptors, clusters of differentiation (CDs), etc. The affinity reagent may be labeled by conventional methods (radioactivity, fluorescence, enzymatic, etc.) to allow detection of the binding thereof at the cell surface. Detection may also be performed using a second, labeled affinity reagent that binds to the first affinity reagent. Any known immunoenzymatic method is suitable (Elisa, Ria, etc.) or by using nanocrystals (Quantum Dot Corporation).

(ii) One or several signal transduction pathway trans-reporter systems, to detect the presence or effect of a cellular element whose production or activity results from activation or expression of the selected gene.

Such a reporter gene construct preferably comprises, a reporter gene (ie, a nucleic acid encoding a product which can be detected) under the control of a transcriptional promoter that is activated in the presence of a molecule involved in the signaling pathway of the selected gene product. Examples of reporter genes include, for instance, any nucleic acid encoding a polypeptide such as green fluorescent protein, β-galactosidase, alkaline phosphatase, luciferase, β-lactamase, or variants or derivatives or homologs thereof. In a particular embodiment, the reporter gene is a β-lactamase gene, ie, any nucleic acid molecule encoding a β-lactamase polypeptide, ie, a polypeptide that can hydrolyse a β-lactam ring. The reporter gene may also encode a selection product (eg, a product conferring antibiotic-resistance), thereby allowing direct positive selection of the cells having the selected phenotype by culture in the presence of said antibiotic (eg, only the activated cells will survive under such conditions). Examples of regulated (or responsive) transcriptional promoters include any promoter comprising one or several copies of a transcription factor binding site such as NFAT, CRE, NF□B, VIP, or JNK, for instance, which have been disclosed in the art. Typical gene reporter constructs of this invention comprise a plasmid, such as pcDNAIII, pUC, etc., in which at least one copy of the reporter gene and promoter has been inserted. The plasmid may further comprise a marker gene, allowing selection of the recombinant cells that contain the reporter construct. Alternatively, the reporter construct may be integrated into the genome of the cells, by any conventional technique, including recombination, transposons, viral integration etc. In preferred embodiments, the reporter construct is stably introduced into the cells using an extrachromosomic vector. More preferably, the construct is stable so that it remains present in the cells after several (preferably 100) cell divisions under selection pressure. The reporter construct (or any vector containing the same) may be introduced into the cells before, during or after mutagenesis, using conventional gene delivery or transfection methods such as electroporation, calcium-phosphate precipitation, cationic lipids-, polymer- or liposome-mediated transfection, viral-mediated infection, etc. Generally, one or several copies of the reporter construct, preferably between 1 and 10 copies are introduced into the cells.

The use of a reporter gene construct is particularly useful to detect and select cells in which a selected gene or gene pathway has been activated, without altering the structure of the selected gene product itself. It is for instance suitable to select cells having an activated membrane receptor, which is active in the absence of a ligand thereof.

(iii) One or several functional assays, to detect biochemical situations that are characteristic of the activation or expression of the selected gene (eg, apoptosis, Anenixin-5 or mitochrondrial staining).

(iv) By positive selection, for instance by using a selection construct comprising a responsive promoter (such as egr-1 promoter) and a marker gene (eg, an antibiotic-resistance gene such as zeocin) and culturing the cells in the presence of a ligand of the selected gene product (eg, a growth factor, such as bFGF) and selecting the cells which survive in the presence of the antibiotic.

In (i)-(iv) above, selection of the cells may be accomplished according to various techniques known in the art, more preferably using a Fluorescence Activated Cell Sorter (FACS) (Parks et al., supra., 1979).

(v) By antibiotic/marker selection driven response (eg, G408, Zeocin, etc.).

These various methods (i)-(v) can be used alone or in combination(s) with each others, to allow an efficient and/or selective selection of the cells having the desired property.

In a specific embodiment, the cells expressing the gene and its protein product of interest are selected using the FACS sorter either using an antibody or by reporter response (eg, GFP, β-lactamase) and grown in the presence of a selection marker.

In another variant, the cells of interest are positively selected by transfecting a promoter construct (eg, egr-1) with a selection marker (eg, Zeocin) which responds to an external stimulus (eg, growth factors either in serum or single growth factor in serum free medium eg, bFGF). When the receptor is expressed after mutagenesis, the cell of interest propagates.

Individual sorted cell clones can then be expanded in culture with or without seeder cells (ie, co-culturing) to expand the cells of interest. Cells are then passed and stocks can be frozen in liquid nitrogen for storing.

Multiple Genes

In the case where multiple selected genes and their products are necessary to achieve a desired phenotype, several cells can be produced separately, each carrying one or some of the activated or expressed selected genes. The cells may then be fused by cell fusion technique to obtain a hybrid cell having the desired phenotype. In this embodiment, the cell populations used separately may be identical (ie, of the same type or origin) or different.

Somatic cell fusion has been described in detail (Koehler G. and Milstein C., Continous cultures of fused cells secreting antibody of predefined specificity, Nature, 1975;256:495) and has been used to identify chromosomes carrying genes controlling drug sensitivity, where fusions were made between a mouse embryonal carcinoma (EC) cell line, F9, and a human bladder cancer cell line, MGH-UI (Wang X., Fox M., Povey S., Masters J. R., Somat. Cell. Mol. Genet., 1998;24(3):165-171).

These fusion techniques are well-known to the skilled artisan and can be used in the present invention.

Selected Gene

The present invention discloses methods of causing expression or activation of a selected gene or gene pathway in a cell. The term gene pathway designates more particularly one or several steps in a cellular mechanism that translate the activation of a molecule into a biochemical effect or phenotype.

Gene activation more preferably results from genetic modifications in the cell's genome that do not alter the structure of the selected gene or gene product itself. Such gene activation include, for instance, causing mutation(s) in the promoter, a negative regulator, a signal transduction protein, a transcription factor, a kinase, a phosphatase, etc., which up regulates the gene of interest or its activity, or its signaling pathway.

For example, nuclear receptors (NR) comprise a family of transcription factors that regulate gene expression (Glass C. and Rosenfeld M., Genes & Development, 2000;14:121-141) and are known to be regulated by phosphorylation (Hammer G. D., Krylova I., Zhang Y., Darimont B. D., Simpson K., Weigel N. L., and Ingraham H. A., Mol. Cell, 1999;3:521-526 and Tremblay A., Tremblay G. B., LaBrie F., and Giguere V., Mol. Cell, 1999; 3:513-519). A nuclear receptor such as estrogen receptor beta can be activated independently of its ligand by recruitment of SRC-1 through phosphorylation of AF-1 (Ingraham et al., supra., 1999), or PPAR alpha is activated and PPAR gamma is inactivated when directly phosphorylated by ERK-1,2. Thus, any mutation, which induces or inhibits these kinases, inversely up regulates these genes without having to mutate the gene of interest.

As indicated before, the selected gene may be any receptor (whether membrane or nuclear), channel, cytoplasmic polypeptide (including enzymes), etc.

In a specific embodiment, the present invention can be used to cause expression or activation of a PTH receptor. The PTH receptor is known to belong to the Gs coupled class of GPCRs. Activation of a receptor of this class leads to an increase in intracellular cAMP concentrations. Any gene in the cell that is regulated by cAMP concentrations is thus effected. It is known that many of these genes contain a cAMP response element (CRE). The CREBP transcription factor, which is activated by binding cAMP, can therefore regulate any gene that has these CRE binding sites. In this way by up-regulating intracellular cAMP concentrations, any gene containing CRE binding sites will be activated. It is also possible to construct a reporter gene, containing one or more CRE binding sites in a row followed by a minimal promoter, and finally with a reporter gene (such as the β-lactamase gene). A reporter gene thus constructed would produce an increase in its β-lactamase gene product upon increases in intracellular cAMP concentrations. When this reporter construct is transfected into cells with the PTH receptor, activation by PTH leads to increases in intracellular cAMP and thus increases in β-lactamase. Thus an increase in the level of β-lactarnase can be determined by observing activity of this enzyme, in particular the hydrolysis of a substrate thereof. This system can be applied to the cell sorter to isolate clones of receptors in a straightforward fashion. An appropriate cell population is transfected with the CRE-β-lactamase reporter gene and treated with PTH. The cells are allowed to stand for an appropriate amount of time and are then loaded with the β-lactamase substrate. These cells can then be examined on a flow cytometer and positive cells isolated as populations or single clones. This method allows the production of cells that express a PTH receptor without cloning the corresponding gene.

In another specific embodiment, the present invention is used to cause activation or expression of GABAb Receptors 1, 2 in a cell. In this regard, another way to identify cells that contain a given receptor is by using antibodies to that receptor in conjunction with flow cytometry. In this case, a prospective cell line can be treated with an antibody to the desired receptor (eg, GABAb). The antibody can be labeled with a fluorescent tag itself, or a second antibody (eg, if the first antibody is a rabbit antibody, the second antibody may be a goat antirabbit antibody) can be labeled with the fluorescent tag and bound to the first antibody. This cell population can then be analyzed by flow cytometry upon mutagenic treatment, and those cells containing receptor (having high fluorescence) can be isolated as populations or single cell clones. Once cell lines are established for GABAb 1 and 2 receptors, the cell lines are fused as described above “Somatic Cell Fusion.” Cells harboring both receptors are selected for activity to GABA. As described above, selection marker and reporter plasmids can be co-transfected into these cell lines, too.

Use for Screening

The present invention can be used to produce cells or cell lines having a desired phenotype. These cells may be used in production, cloning, cell therapy, screening, or any other research purposes. The invention is particularly suited for creating cells with induced gene(s) expression or activity for screening candidate compounds without having to transfect in the cloned gene(s).

In this regard, an object of this invention resides in a method of screening for compounds that modulate a gene or gene product activity in a cell, comprising (i) contacting candidate compounds with said cell, wherein the cell or an ancestor thereof has been exposed to at least a mutagenic agent or condition which causes expression or activation of said gene, and (ii) selecting the compound or compounds that modulate said gene or gene product activity in the cell.

The contacting can be performed in vitro (or ex vivo) in any appropriate support or device, including plate, tube, flask, and the like. Generally, contacting is performed in multi-well plates, allowing multiple assays to be carried out in parallel. Typical supports include microtiter plates, especially the 96-well or 384-well and higher throughput microtiter plate formats, which are easy to manage and easy to illuminate with conventional excitation. Other formats may also be used, including larger microtiter plates or nanotechnologies.

This aspect of the invention is particularly advantageous, since it allows to create cells having desired phenotypes or genotypes with no need for cloning or using of any selected gene.

Other aspects and advantages of the present invention will be disclosed in the following examples, which should be regarded as illustrative and not limiting the scope of the present application.

### EXAMPLE 1

### Activation of Gene Expression or Pathway by Mutagenesis in Whole Animals

Whole animals are individually confined in containers when receiving X-ray to ensure exposure at a constant dose rate. Following irradiation, animals are kept under clean conditions and supplied with acidic drinking water to minimize death due to infection by environmental pathogens before preparing tissues and cells of interest (Selected Methods in Cellular Immunology, 1980, edited by B. B. Mishell and S. M. Shiigisee) and cell sorting according to Parks et al., supra., 1979 to select for cells expressing the desired gene and its protein product. These isolated viable cells are cultured in single 96-wells transfected with an eukaryotic selection marker plasmid (eg, Zeocin, Hygromycin B, G-418, etc.) either with or without feeder cells lacking the resistance markers. Cells are grown for 3 to 7 days without selection before applying the drug of choice for selecting. Drug resistant cells are FACS sorted again using an antibody to the desired gene product and grown in the presence of selection marker before freezing down stocks according to standard conditions.

### EXAMPLE 2

### Activation of Gene Expression or Pathway by Mutagenesis of Cell Suspensions

Single-cell suspensions are washed and resuspended at 5×10⁶ cells/mL in balanced salt solution, and placed in the irradiation chamber before delivering the desired dose of X-ray. After dosing, cells are washed multiple times in balanced salt solution to remove toxic free radicals and their products resulting from irradiation. The cells are transfected with an eukaryotic selection marker plasmid (eg, Zeocin, Hygromycin B, G-418, etc.) and, optionally, with a reporter plasmid (eg, GFP, Luciferase, etc.) using a second selection marker, and cultured either with or without feeder cells lacking these resistance markers. Cells are grown for 3 to 7 days without selection before applying the drug of choice for another 3 to 7 days. Drug resistant cells are FACS sorted (eg, using an antibody to the desired gene product or pathway specific transactivator plasmid “PathDetect” as well as reporter plasmid) and grown in the presence of selection marker before freezing down stocks according to standard conditions (Sambrook J., Fritsch E. F., and Maniatis T., Molecular Cloning, “A Laboratory Manual”, Cold Spring Harbor Laboratory Press, 2nd Edition, 1989). To use the FACS to sort responsive cells, one includes either a beta-lactamase or GFP containing reporter plasmid. The purpose of adding the transactivator is to provide specificity and to amplify the signals produced by the receptor of interest. For example, if one wants to screen compounds interacting with the NK-1 receptor, a cell or cell line is mutagenized, then co-transfected with PFA-2ELK1 (trans-reporter plasmid) and PFR-GFP (reporter plasmid) (see Stratagene PathDetect system handout). Cells harboring both plasmids can be activated and sorted by FACS in the presence of an agonist (eg, Substance P). Once the cell line is established (marker plasmid either on each trans-reporter and reporter) containing the endogenous receptor and the reporter plasmids, one can screen for agonists and antagonists.

What is claimed is:

1. A method of causing expression or activation of a selected gene in a cell, comprising exposing a cell population to at least a mutagenic agent or condition, and selecting the cells in the exposed population that express the selected gene or have an activated selected gene.

2. The method of claim 1, wherein the selected gene encodes a nuclear or membrane receptor.

3. The method of claim 2, wherein the selected gene encodes a receptor for a neurotransmitter, a hormone, a growth factor, a cytokine, or a trophic factor.

4. The method of claim 1, wherein the selected gene encodes a channel.

5. The method of claim 1, wherein the selected gene encodes a cytoplasmic protein.

6. The method of claim 1, for causing overexpression of the selected gene.

7. The method of claim 1, for causing activation of the selected gene in the absence of a natural ligand or activator thereof.

8. The method of claim 1, wherein expression or activation of the selected gene is a result of a mutation in a gene or sequence in the cell that regulates the expression or activity of the selected gene in the cell.

9. The method of claim 1, wherein the cell is a cultivable cell.

10. The method of claim 9, wherein the cell is a mammalian cell.

11. The method of claim 9 or 10, wherein the cell contains the selected gene in its genome, but the gene is not expressed or activated.

12. The method of claim 1, wherein the mutagenesis is performed in vitro, ex vivo, or in vivo.

13. The method of claim 12, wherein the mutagenic agent is a chemical mutagen.

14. The method of claim 12, wherein the mutagenic condition comprises irradiation.

15. The method of claim 1, wherein the selection is performed with an antibody to the selected gene product.

16. The method of claim 1, wherein the selection is performed with a signal transduction pathway reporter system.

17. The method of claim 1, wherein the selection is performed with a functional assay.

18. The method of claim 1, wherein the selection is performed with a marker-driven response.

19. A method of causing expression or activation of at least two selected genes in a cell, comprising:
(i) exposing a first cell population to at least a mutagenic agent or condition and selecting the cells in the first population that express a first selected gene or have an activated first selected gene,
(ii) exposing a second cell population to at least a mutagenic agent or condition and selecting the cells in the second population that express a second selected gene or have an activated second selected gene, and
(iii) fusing the cells selected in (i) and (ii) to produce a cell having two expressed or activated selected genes.

20. A method of causing expression of a selected gene encoding a membrane receptor in a cell, comprising exposing a cell population to at least a mutagenic agent or condition and selecting the cells in the exposed population that bind a ligand of the membrane receptor, said cells expressing the membrane receptor.

21. A method of causing activation of a receptor expressed in a cell so that the receptor is active without a ligand of said receptor, comprising exposing a cell population to at least a mutagenic agent or condition in the absence of a ligand of the receptor, and selecting the cells in the exposed population having an activated receptor.

22. The method of claim 21, wherein the receptor is a membrane receptor.

23. The method of claim 21, wherein the receptor is a nuclear receptor.

24. A method of causing expression or activation of a selected phenotype in a cell, comprising exposing a cell population to at least a mutagenic agent or condition and selecting at least a cell in the exposed population that express the selected phenotype.

25. A method of screening for compounds that modulate a gene activity in a cell, comprising (i) contacting candidate compounds with said cell, wherein the cell or an ancestor thereof has been exposed to at least a mutagenic agent or condition which causes expression or activation of said gene, and (ii) selecting the compound or compounds that modulate said gene activity in the cell.

26. A method of screening for compounds that modulate a gene product activity in a cell, comprising (i) contacting candidate compounds with said cell, wherein the cell or an ancestor thereof has been exposed to at least a mutagenic agent or condition which causes expression or activation of said gene, and (ii) selecting the compound or compounds that modulate said gene product activity in the cell.

27. A cell produced by the method of claim 1.
